(19) United States
(12) Patent Application Publication
(10) Pub. No.: US 2026/0234640 A1
(43) Pub. Date: Aug. 13, 2026

(54) APTAMERS FOR THE DETECTION OF DRUGS, BIOMARKERS, AND SMALL MOLECULES FOR OCULAR APPLICATIONS

(71) Applicant: CENTRE FOR EYE AND VISION RESEARCH LIMITED, Hong Kong (CN)

(72) Inventors: JUEWEN LIU, Waterloo, CA (US); KA-YING WONG, Waterloo, CA (US); YIBO LIU, Waterloo, CA (US); CHAU-MINH PHAN, Waterloo, CA (US); LYNDON JONES, Waterloo, CA (US)

(21) Appl. No.: 19/113,267

(22) PCT Filed: May 20, 2024
(86) PCT No.: PCT/IB2024/054878

§ 371 Date: Mar. 19, 2025

Related U.S. Application Data
(60) Provisional application No. 63/503,153, filed on May 18, 2023.

(51) Int. Cl. C12N 15/115; G01N 33/53

(52) U.S. Cl. CPC C12N 15/115; G01N 33/5308; C12N 2310/16

(57) ABSTRACT

Various embodiments are described herein for utilizing a DNA or RNA aptamer to detect ocular drugs, biomarkers, and small molecules. The DNA or RNA aptamer consists of a binding sequence to selectively and strongly bind to the compound of interest and may be tethered to an easily detectable tag. The tag can be fluorescent, radioactive, or a chromophore. The detection of the compound of interest can then be achieved with simple standard detection methods such as UV-Vis or fluorescence spectrophotometry, or more complex methods such as mass spectrophotometry or electrochemical detection, preferably fluorescence detection or radioactivity detection with high sensitivity.

### FIELD

The various embodiments described herein generally relate to the detection of ophthalmic compounds, including drugs, biomarkers, tear film components, and small molecules used in vision science fields using DNA or RNA aptamers.

### BACKGROUND

The development of new ocular drug delivery methods necessitates sensitive and accurate detection of drugs and other small molecules. However, many of these compounds are difficult, cumbersome, and expensive to detect using current methods such as HPLC (high-performance liquid chromatography) and mass spectrometry.

The detection of tear film biomarkers is of increasing interest for early screening and detection of ocular diseases. However, these biomarkers are often found in very low concentrations and thus are difficult to detect using current methods.

DNA or RNA aptamers are short, single-stranded DNA or RNA molecules that can bind to specific target molecules with high affinity and specificity. Similar to antibodies, these aptamers can be developed to bind to a specific target.

There is still a great need for developing agents and methods that can be used in the detection of drugs, biomarkers, and small molecules for ocular applications with high specificity, high accuracy and low cost.

### SUMMARY OF VARIOUS EMBODIMENTS

Various embodiments for developing DNA or RNA aptamers for the detection of drugs, biomarkers, and other small molecules are provided according to the teachings herein. The DNA or RNA aptamer is composed of a sequence designed to bind to the target compound with high affinity and specificity.

The optimal sequence of the DNA or RNA aptamer can be determined through various screening methods, including but not limited to SELEX (Systematic Evolution of Ligands by Exponential Enrichment) and machine learning-directed aptamer search.

In general, the DNA or RNA aptamer is also tethered to a detectable tag, such as a fluorescent, electrochemical active or radioactive molecule or a chromophore. The binding of the DNA or RNA aptamer-tag to the target enables sensitive quantification of the target.

In at least some embodiments, the DNA or RNA aptamer may have more than one binding site to a single compound.

In at least some embodiments, the DNA or RNA aptamer may have binding affinity to more than one target compound.

In at least some embodiments, the DNA or RNA aptamer may also bind to other small molecules that are not drugs or biomarkers.

In at least some embodiments, the DNA or RNA aptamer might be immobilized on a surface, such as a testing line, magnetic beads, microplates, nanoparticles, or a microchip as high-throughput immobilized aptamer-based biosensors.

In at least some embodiments, a complementary strand to DNA or RNA aptamer is also designed with a quencher for strand displacement-based detection.

In at least some embodiments, the labelled-free DNA or RNA aptamer is also designed for target detection. The binding of the DNA or RNA aptamer might also be detected by fluorescent dyes, such as ThT, SYBR green, EvaGreen, or TaqMan probes.

The DNA or RNA aptamer may also be used in combination with other detection methods, such as mass spectrometry or electrochemical detection, to enhance the sensitivity and selectivity of the assay.

The use of DNA or RNA aptamer for ocular application may also involve optimization of assay conditions, such as pH, temperature, and incubation time, to maximize the binding affinity and specificity of the aptamers towards the target molecule.

Other objects, features, advantages and aspects of the present application will become apparent to those skilled in the art from the following description and appended claims. It should be understood, however, that the following description, appended claims, and specific examples, while indicating preferred embodiments of the application, are given by way of illustration only. Various changes and modifications within the spirit and scope of the disclosed invention will become readily apparent to those skilled in the art from reading the following.

### BRIEF DESCRIPTIONS OF DRAWINGS

For a better understanding of the various embodiments described herein and to show more clearly how these various embodiments may be carried into effect, reference will be made, by way of example, to the accompanying drawings, which show at least one example embodiment, and which are now described.

FIG. 1 shows examples of aptamers (A) binding to specific targets (B), including but not limited to biomarkers, small molecules (drugs, metabolites, toxins, environmental pollutants), proteins, nucleic acids, cells, or tissues, to (C) form various aptamer-target complexes.

FIG. 2 shows the detection of a target using an aptamer, including but not limited to strand displacement (A), fluorescence resonance energy transfer (B), aptamer beacon (C), or fluorescent dyes (D).

FIG. 3 shows the mechanism of detecting an ocular drug (atropine) in tears using an aptamer-based biosensor.

FIG. 4 shows the binding affinities of aptamers to timolol malate and atropine by ITC.

FIG. 5 shows the calibration curve and the limit of detection for atropine using UV-vis and aptamer-based fluorescence biosensor.

### DETAILED DESCRIPTION OF EMBODIMENTS

Various processes will be described below to provide an example of at least one embodiment of the claimed subject matter. No embodiment described below limits any claimed subject matter, and any claimed subject matter may cover processes or systems that differ from those described below. The claimed subject matter is not limited to processes or systems having all of the features of any process or system described below or to features common to multiple or all of the processes or systems described below. It is possible that a process or system described below is not an embodiment of any claimed subject matter. Any subject matter that is disclosed in a process or system described below that is not claimed in this document may be the subject matter of another protective instrument, for example, a continuing patent application, and the applicants, inventors, or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures, and components have not been described in detail so as not to obscure the embodiments described herein. Also, the description is not to be considered as limiting the scope of the embodiments described herein.

It should be noted that terms of degree such as “substantially”, “about” and “approximately” as used herein mean a reasonable amount of deviation of the modified term such that the end result is not significantly changed. These terms of degree may be construed as including a certain deviation of the modified term if this deviation would not negate the meaning of the term it modifies.

Furthermore, the recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation up to a certain amount of the number to which reference is being made if the end result is not significantly changed.

As used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

As used herein, the term “a” or “an” is intended to mean “one or more” (i.e., at least one) of the grammatical object of the article. Singular expressions, unless defined otherwise in contexts, include plural expressions. By way of example, “an element” means one element or more than one element.

As used herein, unless otherwise noted, the term “comprise”, “include” and “including” will be understood to imply the inclusion of a stated step or element or group of steps or elements but not the exclusion of any other step or element or group of steps or elements which do not affect the end result. The terms “comprising”, “comprises” and “comprised” may also include the term “consisting essentially of” and “consisting of”.

Described herein are various example embodiments for a DNA- or RNA-aptamer for the detection of ocular drugs, biomarkers, and small molecules and associated methods for fabrication thereof and testing in a variety of applications.

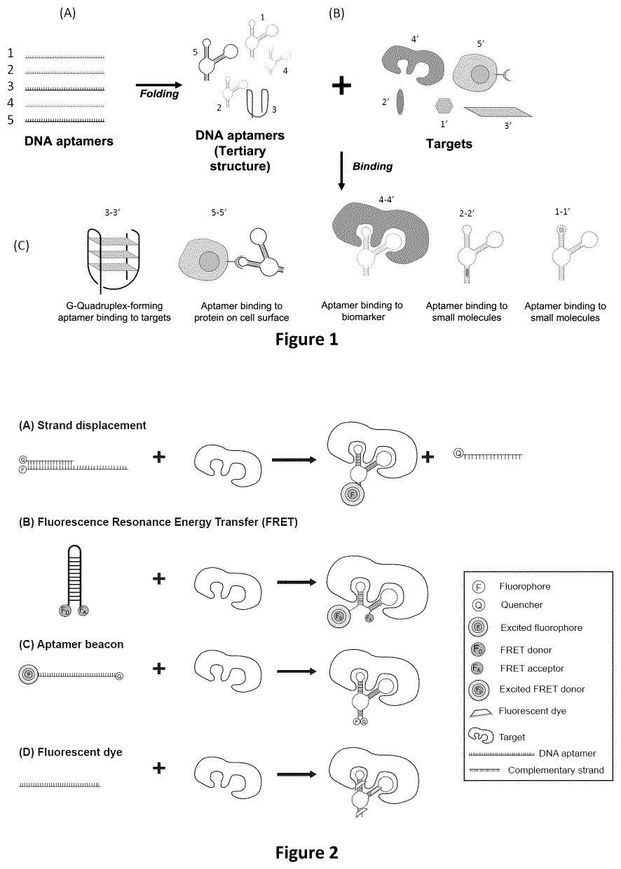

DNA or RNA aptamers are short, single-stranded DNA or RNA molecules that can bind to specific target molecules with high affinity and specificity. As illustrated in FIG. 1, aptamers may have diverse primary structures, such as aptamers 1-5 with different nucleotide sequences (A), and may fold into diverse three-dimensional structures, i.e., with diverse tertiary structures (B). Targets for the aptamers include but not limited to biomarkers, small molecules (drugs, metabolites, toxins, environmental pollutants), proteins, nucleic acids, cells, or tissues (such as targets 1′~5′). When aptamers are exposed to their corresponding specific targets under proper conditions, they bind to corresponding targets in three dimensional shapes with high affinity and specificity (C).

The aptamer selection process utilizes SELEX (Systematic Evolution of Ligands by Exponential Enrichment) to select aptamers that have high specificity and affinity to the target. SELEX has been a gold-standard strategy for the generation of nucleic acid aptamers. The selection cycle, whether for DNA or RNA sequences, on proteins, on cellular levels, or in living animals, requires three pivotal steps: (i) incubating a target with a library containing randomized sequences, (ii) partitioning bound sequences from non-bound sequences, and (iii) recovering and PCR amplifying the bound sequences.^{[1]} Aptamer selected from various materials can be used in the present application as long as the selected materials are eye-related and can be used in detection for ocular application.

Aptamers can be selected by, but not limited to, the library immobilization method or target immobilization method.^{[1,2]} In contrast to traditional SELEX method, our library capture approach, offers enhanced robustness and increase the success rate. The whole process involves several iterative rounds of selection and amplification, as outlined in the 7 steps below.
(1) Library synthesis: A library of randomized nucleic acid sequences, typically 10¹³-10¹⁵ different sequences and 25-60 bases random library with flanking sequence, is synthesized by combinatorial chemistry. The library can be made up of DNA or RNA sequences, including chemically modified nucleic acids, depending on the desired application.
(2) Library immobilization: the single-stranded library is first annealed with a biotinylated capture strand in the selection buffer. The double-stranded library is immobilized on a solid support, such as streptavidin-coated raisin or magnetic beads, or a microplate. This can be done through covalent attachment or non-covalent interactions.
(3) Selection: the target is incubated with the immobilized library under conditions that favour the formation of the aptamer-target complex. Unbound sequences are washed away, and the bound sequences are eluted and amplified by polymerase chain reaction (PCR).
(4) Counter-selection: The amplified pool of sequences is subjected to a counter-selection step to remove any sequences that bind to molecules other than the target molecule. This is typically done by incubating the pool with a non-specific target molecule that is similar to the target molecule but lacks the desired binding properties.
(5) The selected sequences are used as the starting point for the next round of selection and amplification. The number of rounds can vary depending on the complexity of the target molecule and the desired affinity and specificity of the aptamer.
(6) Aptamer characterization: the selected sequences are sequenced to identify the aptamer candidates. These candidates are further characterized for their binding affinity and specificity using techniques such as isothermal titration calorimetry (ITC), surface plasmon resonance (SPR), electrophoretic mobility shift assays (EMSA), flow cytometry or enzyme-linked oligonucleotide assay (ELONA).
(7) Aptamer optimization: The identified aptamer candidates are then optimized through various modifications, including truncation, sequence modification, and chemical modification, to further enhance their affinity and specificity towards the target molecule.

The success or failure of SELEX depends on a variety of different factors, including the structural diversity offered by the oligonucleotide library, the effective removal of nonbinders and weak binders during the selection step, and the selective enrichment of high-affinity binders during the amplification step, ideally without by-products. The selection step should be designed to mimic the desired future application of the aptamer as closely as possible in order to avoid downstream difficulties.^{[2]} Among others, the selection buffer should provide optimal conditions for the formation of the aptamer-target complex while minimizing non-specific binding. The selection buffer typically, but is not limited to, contains buffer solution, such as phosphate-buffered saline (PBS), Tris-buffered saline (TBS), or HEPES-buffered saline (HBS), blocking agents such as bovine serum albumin (BSA) or casein and ions such as magnesium ions, calcium ions, potassium ions, sodium ions. One of the significant novel points of the current application is the utilization of the selection method for real ocular drug detection. In some preferred embodiments, the selection buffer is modified from Tris to PBS to better replicate ocular conditions.

In some embodiments, the library is incubated directly with targets such as cells and tissues without immobilization.

In some embodiments, capillary electrophoresis-SELEX, random primer-initiated polymerization chain reaction-SELEX, cell-SELEX, microfluidic-based SELEX, magnetic-bead based SELEX, and high-throughput sequencing-based SELEX may be used.^{[4]}

In some embodiments, machine learning directed aptamer search may also be used.^{[3]} Computer-aided prediction of aptamer sequences focusing on primary sequence alignment and motif comparison has been developed by taking conserved hairpin with highly variable sequence into consideration and with three scores based on sequence abundance, stability, and structure, respectively.

In contrast to the traditional SELEX method, our library capture approach, offers enhanced robustness and increase the success rate. Furthermore, the aptamer-based biosensor is demonstrated to have greater sensitivity and specificity compared to conventional UV-vis methods when detecting small molecules. Furthermore, the invention's cost-effectiveness, rapid detection capabilities, and simplified sample preparation procedures enhance its practicality and usability compared to HPLC-MS. Overall, these features make the invention straightforward to implement and reproduce.

In some embodiments of the present disclosure, timolol maleate-specific aptamers with high binding affinity, specificity and sensitivity to timolol maleate, such as those as set forth by the nucleotide sequence of any one of SEQ ID NOs: 1-11 or having at least 80%, at least 90%, at least 95%, at least 98%, at least 99% sequence identity to any one of SEQ ID NOs: 1-11, are provided by the modified method of the present disclosure. In some embodiments, atropine-specific aptamers with high binding affinity, specificity and sensitivity to atropine, such as those as set forth by the nucleotide sequence of any one of SEQ ID NOs: 12-21 or having at least 80%, at least 90%, at least 95%, at least 98%, at least 99% sequence identity to any one of SEQ ID NOs: 12-21, are provided by the modified method of the present disclosure.

The detection of a target using aptamers is described in detail below.

The aptamer-based biosensors are used as recognition elements in a variety of detection methods for targets. As used herein, “target” can be any biomarkers, small molecules (drugs, metabolites, toxins, environmental pollutants), proteins, nucleic acids, cells, or tissues.

In some embodiments, the aptamer is tagged with a radioactive, fluorescent, or chromophore tag. A short complimentary stand to aptamer can be tagged with a quencher.

The targets can be detected by aptamer-based biosensors where the transducer could be optical transducers such as fluorescent readout, radioactivity intensity, colorimetric spectroscopy or polarization, electrochemical transducers such as potentiometry, amperometry, or impedance spectroscopy and magnetic transducers such as magnetic relaxation switches or magnetic resonance imaging. FIG. 2 illustrates examples for the detection of a target using an aptamer, including but not limited to strand displacement (A), fluorescence resonance energy transfer (B), aptamer beacon (C), or fluorescent dyes (D). Upon binding of an aptamer to its specific target, (A) a quencher binding to the aptamer, which quenches the emission of a fluorophore also attached to the aptamer, is replaced by the target, and enables fluorescence-based quantification of target binding; (B) the dipole-dipole interaction between a donor and an acceptor both attached to the aptamer is changed (such as due to the distance change between the donor and the acceptor), which enables FRET detection by the appearance of sensitized and excited fluorescence; (C) donor-quencher pair (such as fluorescent dyes, quantum dots, carbon-based materials, and metallic nanoparticles) acting as an optical on/off switch on the aptamer changes, which enables visualization of the specific binding; and/or (D) a fluorescent dye can further bind to the aptamer-target complex to produce detectable fluorescent signals. Other detection ways are known in the art and can be selected according to the need in practice.

The target to be detected can be in collected tears, extracted animal tissues, and extracted cell lysates or culture medium. Tear fluid forms the outermost layer of the ocular surface and its characteristics and composition have been connected to various ocular surface diseases. As tear proteomics enables the non-invasive investigation of protein levels in the tear fluid, it has become an increasingly popular approach in ocular surface and systemic disease studies.^{[5]} Hence, tear fluid is an important target of aptamers for ocular application of the present disclosure.

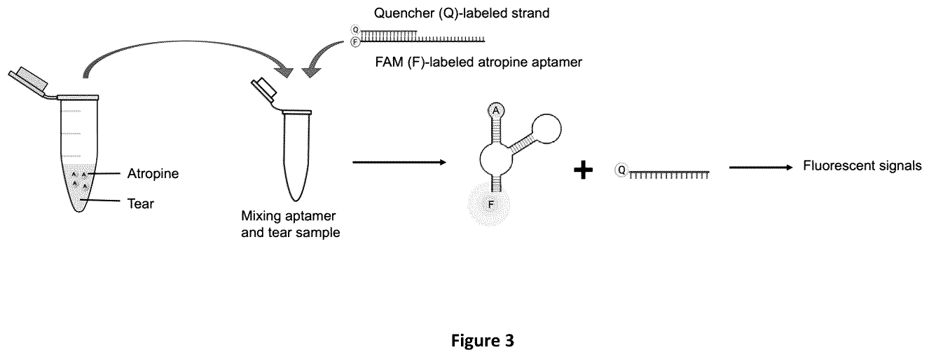

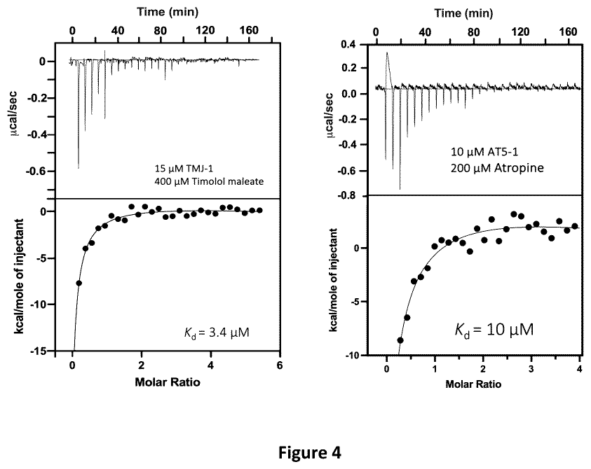

FIG. 3 shows the mechanism of detecting an ocular drug (atropine) in tears using an aptamer-based biosensor. Atropine-specific aptamers can be added into and mixed with a sample, preferably tear sample, to be detected comprising or suspected to comprise atropine. Upon specific binding of the aptamers to atropine, the quencher-labeled strand leaves the FAM-labeled atropine-specific aptamer and fluorescent signals are produced.

The possible targets of aptamer for ocular application include but are not limited to those listed in table 1.^{[5, 6]} In some embodiments, the target of aptamer is an ophthalmic drug, such as selected from a drug for glaucoma care, corticosteroids, antibiotics, combination drugs, for ocular surface care, allergy drugs, for shingles therapy, and for vision insights.

TABLE 1
Examples for DNA aptamer targets for ocular application

| TYPE/CLASS OF DRUGS | DRUGS |
|---|---|
| TOPICAL ANTIBIOTIC DRUGS | |
| I. FLUOROQUINOLONES | Besifloxacin, Ciprofloxacin, |
| | Moxifloxacin, Ofloxacin, Gatifloxacin |
| II. AMINOGLYCOSIDES | Tobramycin, Gentamicin |
| III. OTHER ANTIBIOTICS | Azithromycin, Erythromycin, Bacitracin |
| OCULAR ALLERGY | Ketorolac tromethamine, Ketotifen |
| MEDICINES | fumarate, Bepotastine besilate, |
| | Epinastine HCl, Emedastine |
| | difumarate, Alcartadine, Azelastine |
| | hydrochloride, Olopatadine, |
| | Hydrochloride, ketotifen fumarate |
| CHRONIC CARE PRODUCTS | Nedocromil sodium, Iodoxamide |
| | tromethamine, Cromolyn sodium |
| TOPICAL CORTICOSTEROID | Difluprednate, Loteprednol |
| DRUGS | etabonate, Prednisolone acetate, |
| | Prednisolone sodium |
| | phosphate, Dexamethasone |
| MODERATEAND LESSER | Loteprednol etabonate, |
| STRENGTH STEROIDS | Fluorometholone acetate, |
| | Fluorometholone alcohol, |
| | Prednisolone acetate |
| NON-STEROIDAL ANTI- | |
| INFLAMMATORY DRUGS | |
| I. ORAL | Ibuprofen, Naproxen, |
| | Celecoxib, Diclofenac |
| II. TOPICAL | Ketorolac, Bromfenac, |
| | Diclofenac, Nepafenac |
| DRY EYE MEDICATION | Cyclosporine, Lifitegrast |
| TOPICAL GLAUCOMA DRUGS | |
| I. BETA BLOCKERS | Levobunolol hydrochloride, Betaxolol |
| | hydrochloride, Timolol maleate |
| II. PROSTAGLANDIN | Bimatoprost, Travoprost, |
| ANALOGS | Latanoprost, Tafluprost |
| III. ALPHA AGONISTS | Brimonidine |
| IV. CARBONIC | Brinzolamide, Dorzolamide |
| ANHYDRASEINHIBITORS | |
| TOPICAL MYOPIA DRUGS | Atropine |

One notable innovation introduced by this invention is the development of aptamer biosensors designed explicitly for ocular applications, utilizing either fluorescent or radioactive signals. For instance, conventional UV-vis analysis may encounter interference from various molecules or hydrogel materials absorbing at 257 nm, potentially compromising the accuracy of atropine detection. Aptamer-based methods offer enhanced specificity since the aptamer can selectively bind to its target, resulting in lower detection limits compared to UV-vis. Moreover, when compared to HPLC-MS, aptamer-based detection is generally less complex and more cost-effective in terms of both materials and equipment usage.

In addition, Wang Z. et al. discloses an aptamer-based graphene affinity nanobiosensor for the detection of inflammatory markers in eye. The nanobiosensor is a graphene field-effect transistor, in which a nucleic acid aptamer and a biomolecule-permeable polyethylene glycol (PEG) nanolayer are immobilized on the graphene surface.^{[7]} Wang Y. et al. discloses aptamer-based liquid crystal film on a glass support for the detection of kanamycin.^{[0]} However, aptamer attachment on a surface is needed for the detections in both articles. To the contrast, the simple detection methods of the present disclosure can be carried out with or without aptamer attachment to a surface.

As a particular example, the present application of this invention could be used in detecting atropine in tears to monitor drug delivery efficiency. As a further particular example, the present application of this invention could be used in detecting timolol malate in tears to monitor drug delivery efficiency or the maintenance of the drug in target. These examples are provided merely for illustration purposes and shall not be interpreted to limit the scope or content of the present invention in any way.

### EXAMPLES

The present invention is further illustrated by the following examples. These examples are provided merely for illustration purposes and shall not be interpreted to limit the scope or content of the present invention in any way.

Publications cited herein and the materials for which they are cited are hereby specifically incorporated by reference in their entireties. All reagents, unless otherwise indicated, were obtained commercially. All parts and percentages are by weight unless stated otherwise. An average of results is presented unless otherwise stated. The abbreviations used herein are conventional, unless otherwise defined.

### Materials and Methods

### 1. Materials

DNA sequences used for the selection and sensing experiments were synthesized by commercials, like Integrated DNA Technologies. Streptavidin coated agarose resin was purchased from Thermo Scientific. 3k and 10k Ultra-0.5 centrifugal filter units were purchased from Millipore-Sigma. Micro bio-spin chromatography columns and SsoFast EvaGreen supermix were from Bio-Rad. dNTP mix and Taq DNA polymerase with ThermoPol buffer were from New England Biolabs. Atropine and timolol maleate were purchased from Sigma-Aldrich.

### 2. Methods

### 2.1 Capture-SELEX for Selection of Aptamers

### 2.1.1 Preparation of DNA Library

The DNA library contained a 30-nucleotide randomized region flanked by primer binding sequences. The length of the random nucleotides could be varied from 10 up to 60.

### 2.1.2 Preparation of Target Solution, Selection Buffer, and Separation Buffer

Target solution was prepared by dissolving the target in selection buffer. Two buffers were used. The first one was selection buffer containing 1×PBS, 1 mM MgCl₂, 5 mM KCl and 1 mM CaCl₂) at pH 7.5 for DNA binding to the target. Another buffer was separation buffer containing 1×PBS and 5 mM KCl at pH 7.5 for strand separation. 8 mM EDTA was used to dissociate the bound DNA from the target. The concentration of salt and EDTA could be optimized for different target.

### 2.1.3 Procedures of Capture-SELEX

Prior to commencing aptamer selection target stock solution was prepared using the selection buffer. Initially, the DNA library underwent annealing with a biotinylated capture strand in the selection buffer, followed by cooling to room temperature and subsequent storage at −20° C. Streptavidin-coated agarose resin was then introduced into a micro bio-spin chromatography column and subjected to six wash cycles with selection buffer to eliminate any residual preservatives. The prepared biotin-DNA complex was subsequently introduced into the agarose resin and underwent multiple loading cycles, exceeding six. This was followed by 12 wash cycles with selection buffer to remove non-bound or weakly bound sequences. The target working solution was applied to the library at room temperature. Upon binding of the aptamer to the target, the aptamer-target complex was released from the resin and eluted with target solution. The eluted DNA was collected via gravity flow. The eluent was then concentrated and further purified using a 3k filter for PCR, with the PCR products undergoing additional concentration and purification using a 10k filter. The purified PCR products were loaded onto clean agarose resin and subjected to another wash with separation buffer. The agarose resin, now bound with DNA, was treated with 0.2 M NaOH, and the eluent was concentrated and purified once more using a 3k filter. Finally, the concentration of the collected single-stranded DNA was confirmed and utilized for the subsequent round of selection, typically repeated at least 12 times. Sequencing was performed on the sequences collected during the final selection round.

### 2.2 Isothermal Titration Calorimetry (ITC)

ITC was performed using a MicroCal VP-ITC. All aptamers and target molecules were prepared in selection buffer. The DNA was annealed, cooled to room temperature, and degassed for 5 minutes before loading. The target solution was then loaded into the syringe and the aptamer was injected into the cell chamber. Apart from an initial injection of 0.5 μL, 10 μL of the target solution was titrated into the cell each time over a 20-second duration, for a total of 28 injections at 25° C. The interval between injections was set to 360 seconds. The syringe stirring speed was maintained at 90 rpm (low speed) to prevent bubble formation, which was critical for the success of the ITC experiment. The binding constant was determined by fitting the titration curve to a one-site binding model using Origin software.

### 2.3 Aptamer-Based Biosensor Assay

In a standard procedure, a mixture was prepared comprising 500 μL of PBS supplemented with 5 mM MgCl₂, alongside 1 μM of aptamer and 10 UM of thioflavin T (ThT). This mixture was subsequently transferred into a quartz cuvette. Sequentially, atropine, initially present at a concentration of 6000 ppm or 20.7 mM, was incrementally added to the solution until achieving a final concentration of 400 μM within the reaction. Throughout the titration process, fluorescence measurements were taken with an excitation wavelength set at 400 nm, while emission was monitored between 460 nm and 550 nm. The recorded fluorescence values at 490 nm were specifically utilized for subsequent analyses. ThT demonstrates fluorescence upon binding to DNA. As atropine was introduced into the solution, displacing ThT from DNA binding sites, the fluorescence values exhibited a decrease. Consequently, the decline in fluorescence served as an indicator of the binding interaction between the aptamer and atropine. The experiment's limit of detection (LOD) was determined employing the formula [LOD=3.3σ/S], where σ represents the standard deviation of the blank and S denotes the slope of the calibration curve.

### 2.4 Ultraviolet-Visible (UV-Vis) Spectroscopy

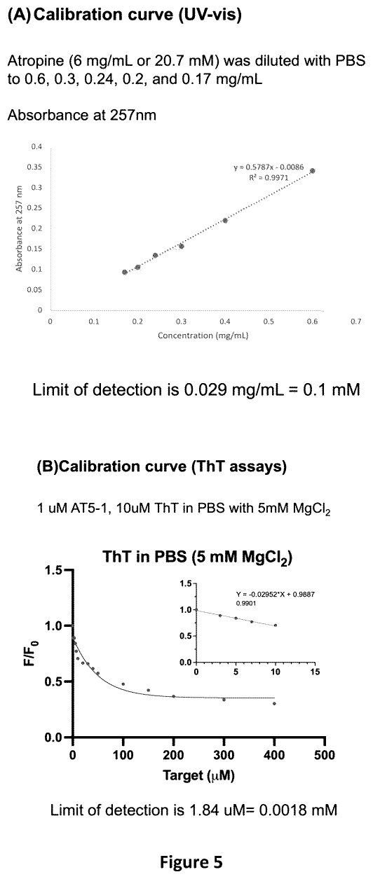

Initially, a 6000 ppm (6 mg/ml or 20.7 mM) atropine solution was diluted to varying concentrations of 0.6, 0.3, 0.24, 0.2, and 0.17 mg/mL with PBS. Subsequently, 100 μL aliquots of each solution were transferred into separate cuvettes for UV-vis analysis. UV absorbance readings were recorded across the spectrum ranging from 200 to 600 nm, with specific focus on the absorbance at 257 nm for the calibration curve (FIG. 5(A)). Lastly, the limit of detection (LOD) was calculated using the formula [LOD=3.3σ/S], where σ represented the standard deviation and S denoted the slope.

### Example 1. Aptamer Selection for Timolol Maleate and Atropine

Aptamer selection for timolol maleate and atropine was conducted through Capture-SELEX following procedures disclosed above in section 2.1.

Tables 2 and 3 show the analyzed sequencing results. The most abundant sequences underwent ITC analysis to confirm their binding to their respective targets (FIG. 4).

TABLE 2
Top 11 sequences from the timolol maleate-SELEX
in the sequencing results of the enriched pool

| | Sequence (5′-3′) | Reads |
|---|---|---|
| Population 1 | | 91% |
| TMJ-1 | GACGACGGGGTTCCTCCGCGGTGGGGCGTTTACGCGGTCGTC | 10448 |
| TMI-2 | GACGACTAGTGGTTCCTCCGCGGTGGGGCTTGATAAGTCGTC | 2080 |
| TMJ-3 | GACGACGGGTTCCTCCGCGGTGGGGTTCCTTTGAACGTCGTC | 1182 |
| TMJ-7 | GACGACGGGGTTCCTCCGCGGTGGGGCTTGATAAGTCGTC | 56 |
| Population 2 | | 1.9% |
| TMJ-5 | GACGACGGGATCCTCCGCGGTGGGGTAACTTGATACGTCGTC | 256 |
| TMJ-8 | GACGACGGGATCCTCCGCGGTGGGGTAACTTGGTACGTCGTC | 38 |
| Population 3 | | 0.2% |
| TMJ-10 | GACGACGTCGGTCGCTCCGCGCTGGGGTTTTAGTCGTCGTC | 22 |
| TMJ-11 | GACGACGGTCGCTCCGCGCTGGGGTGAAATTATTCAGTCGTC | 9 |
| Population 4 | | 4% |
| TMJ-4 | GACGACTGGGGTTCCTACGCGGTGGGTCTTTAGCTGGTCGTCC | 617 |
| Population 5 | | 0.5% |
| TMJ-6 | GACGACGGGTTTGTTATGAGACATTGCCTAAAGGTCGAAGCTGT | 78 |
| Population 6 | | 0.28% |
| TMJ-9 | GACGACGGATGAAGGGGGATTTTCGCCGTTTGCTATGAAGGAGT | 24 |

Note:
Sequences from top to bottom are designated as SEQ ID NOs: 1-11, respectively. Read refers to the DNA sequence from one fragment (a small section of DNA). The percentage is determined by calculating (the number of reads that correspond to the specified sequences/ the total number of reads obtained in sequencing). This calculation assessed the relative abundance of the targeted sequences within the samples.

TABLE 3
Top 10 sequences from the atropine-SELEX
in the sequencing results of the enriched pool

| | Sequence (5′-3′) | Reads (%) |
|---|---|---|
| AT5-1 | GACGACGCCCGTTCCAAACTTGTCGTGCTCTTTTGTGTCGTC | 24,534 |
| | | (40.4%) |
| AT5-2 | GACGACGGAGTTGCAAACGTCGCGGTAGGTTCCCCAGTCGTC | 20,925 |
| | | (34.5%) |
| AT5-3 | GACGACGCAACAGTGCAATACAATGGAACGCGACCTGTCGTC | 305 |
| | | (0.5%) |
| AT5-4 | GACGACCATGAGTCCCCTAAGTTATTAAGGGTGTTCGTCGTC | 133 |
| | | (0.22%) |
| AT5-5 | GACGACGTGGAGCGCATGTGTTGCTCATCTCCGATAGTCGTC | 124 |
| | | (0.2%) |
| AT5-6 | GACGACGCAACAGGTAACAACTAGATACCGCGACCTGTCGTC | 116 |
| | | (0.19%) |
| AT5-7 | GACGACGGCATGGCGCTTGTATTGCTCAACCTTGACGTCGTC | 87 |
| | | (0.14%) |
| Population 1 | | |
| AT5-8 | GACGACGATCAGGAGGGTTCCGGTGGAGGTAGCTAAGTCGTC | 74 |
| | | (0.12%) |
| AT5-9 | GACGACGATCAGGATGTTGTTATAACCGGTCGCTAAGTCGTC | 69 |
| | | (0.11%) |
| AT5-10 | GACGACGACCAGAAGGAATCCAAGGGAGGTGGCTAAGTCGTC | 67 |
| | | (0.11%) |

Note:
Sequences from top to bottom are designated as SEQ ID NOs: 12-21, respectively. Read refers to the DNA sequence from one fragment (a small section of DNA). The percentage is determined by calculating (the number of reads that correspond to the specified sequences/ the total number of reads obtained in sequencing). This calculation assessed the relative abundance of the targeted sequences within the samples.

The results suggest that the library capture SELEX approach of the present application can be used in the effective selection of aptamers, and offers enhanced robustness and increased success rate.

### Example 2. Aptamer-Based Biosensor for Atropine

To assess the detection efficiency of the aptamers for atropine, we employed ThT as a probe to demonstrate the aptamer's binding to atropine following procedures disclosed above in section 2.3. In the absence of the target, ThT binds to the DNA, resulting in green fluorescence detectable via fluorescence spectroscopy. We titrated atropine into the aptamer. The binding of atropine to the aptamer caused a decrease in fluorescence due to displacement of ThT. The limit of detection was subsequently determined. This limit was then compared to that obtained from traditional UV-vis methods following procedures disclosed above in section 2.4.

As shown in FIG. 5, the detection limit of UV-vis was 0.1 mM (see (A)), whereas the aptamer-based ThT biosensor exhibited a detection limit of 0.00018 mM (see (B)).

The results demonstrate that the aptamer-based biosensor has greater sensitivity and specificity compared to conventional UV-vis methods for detecting small molecules. These features make the invention straightforward to implement and reproduce.

The present invention is not to be limited in scope by the embodiments disclosed herein, which are intended as single illustrations of individual aspects of the invention, and any that are functionally equivalent are within the scope of the invention.

Various modifications to the compositions and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

### REFERENCES

1. Ni, S., et al., Recent Progress in Aptamer Discoveries and Modifications for Therapeutic Applications. ACS Appl Mater Interfaces, 2021. 13 (8): p. 9500-9519.
2. Kohlberger, M. and G. Gadermaier, SELEX: Critical factors and optimization strategies for successful aptamer selection. Biotechnol Appl Biochem, 2022. 69 (5): p. 1771-1792.
3. Perez Tobia, J., et al., Machine Learning Directed Aptamer Search from Conserved Primary Sequences and Secondary Structures. ACS Synthetic Biology, 2023. 12 (1): p. 186-195.
4. Zhuo, Z., et al., Recent Advances in SELEX Technology and Aptamer Applications in Biomedicine. International journal of molecular sciences, 2017. 18 (10): p. 2142.
5. Patrick, R., R. Thomas, and P. Vollmer, 2019 Ophthalmic Drug Guide, in Review of Optometry 2019.
6. Nättinen, J., et al., Clinical Tear Fluid Proteomics-A Novel Tool in Glaucoma Research. Int J Mol Sci, 2022. 23 (15).
7. Wang Z, Dai W, Yu S et al. Towards detection of biomarkers in the eye using an aptamer-based graphene affinity nanobiosensor. Talanta 2022; 250:123697.
Wang Y, Wang B, Shen J et al. Aptamer based bare eye detection of kanamycin by using a liquid crystal film on a glass support. Mikrochimica acta (1966) 2017; 184:3765-3771.

1. A DNA or RNA aptamer for detection of an ocular target compound, wherein the aptamer comprises:
a DNA or RNA sequence that selectively binds to the ocular target compound; and
is or is not tethered to a detectable tag, and
wherein the detection is a fluorescence detection or radioactivity detection.

2. The DNA or RNA aptamer according to claim 1, wherein the aptamer is capable of binding to a target compound selected from the group consisting of a drug, biomarker, and other small molecule for ocular application.

3. The DNA or RNA aptamer according to claim 1, wherein the aptamer is capable of binding to the target compound at multiple sites.

4. The DNA or RNA aptamer according to claim 1, wherein the aptamer is capable of binding to multiples of the same target compound.

5. The DNA or RNA aptamer according to claim 1, wherein the aptamer is capable of binding to two more different target compounds simultaneously.

6. The DNA or RNA aptamer according to claim 1, wherein the tag is radioactive, fluorescent, or is a chromophore; and/or
wherein the detection is based on one or more selected from strand displacement, fluorescence resonance energy transfer, aptamer beacon and fluorescent dyes.

7. The DNA or RNA aptamer according to claim 1, wherein the aptamer is attached to or immobilized to a surface; or, is not attached or immobilized to a surface.

8. The DNA or RNA aptamer according to claim 1 used for detecting the release of a drug on the eye or from a biomaterial; detecting biomarkers from the tear film; and/or detecting targets on the eye surface.

9. The DNA or RNA aptamer according to claim 1, wherein the aptamer is a timolol maleate-specific aptamer or an atropine-specific aptamer.

10. The DNA or RNA aptamer according to claim 9, wherein the timolol maleate-specific aptamer is selected from aptamers as set forth by the nucleotide sequence of any one of SEQ ID NOs: 1-11 or having at least 80%, at least 90%, at least 95%, at least 98%, at least 99% sequence identity to any one of SEQ ID NOs: 1-11; and/or wherein the atropine-specific aptamer is selected from aptamers as set forth by the nucleotide sequence of any one of SEQ ID NOs: 12-21 or having at least 80%, at least 90%, at least 95%, at least 98%, at least 99% sequence identity to any one of SEQ ID NOs: 12-21.

11. The DNA or RNA aptamer according to claim 1 immobilized on a surface, such as a testing line, magnetic beads, microplates, nanoparticles, or a microchip as high-throughput immobilized aptamer-based biosensors.

12. The DNA or RNA aptamer according to claim 1, wherein the aptamer is for chromophore detection of an ocular target compound.

13. A method for detecting an ocular target compound in a sample, wherein the method comprises contacting the sample with a DNA or RNA aptamer targeting the ocular target compound, wherein the aptamer comprises: a DNA or RNA sequence that selectively binds to the ocular target compound of interest; and is or is not tethered to a detectable tag; and wherein the detection is a fluorescence detection or radioactivity detection.

14. The method of claim 13, wherein the sample is an in vivo, in vitro or ex vivo sample; and/or wherein the sample is an ocular sample, such as from eye or an ocular region in eye, tear drops, tear film, eye discharge; and/or
wherein the target compound is one or more selected from biomarkers, drugs, metabolites, toxins, environmental pollutants, proteins, nucleic acids, cells, or tissues; and/or
wherein the detection is selected from a group consisting of: drug detection in tears, animal tissues, and human samples to monitor the drug delivery efficacy; biomarker detection in eyes for disease diagnosis and disease progression; biomarker detection for eye imaging.

15. The method of claim 13, wherein the detection of the target compound is a detection based on strand displacement, fluorescence resonance energy transfer, aptamer beacon, or fluorescent dyes; or by chromophore detection.


SEQUENCE LISTING (21 sequences)

SEQ ID NO: 1 - DNA, 42 bp, synthetic construct
  misc_feature 1..42: note = Artificial aptamer TMJ-1
  source 1..42: mol_type = other DNA
gacgacggggttcctccgcggtggggcgtttacgcggtcgtc

SEQ ID NO: 2 - DNA, 42 bp, synthetic construct
  misc_feature 1..42: note = Artificial aptamer TMJ-2
  source 1..42: mol_type = other DNA
gacgactagtggttcctccgcggtggggcttgataagtcgtc

SEQ ID NO: 3 - DNA, 42 bp, synthetic construct
  misc_feature 1..42: note = Artificial aptamer TMJ-3
  source 1..42: mol_type = other DNA
gacgacgggttcctccgcggtggggttcctttgaacgtcgtc

SEQ ID NO: 4 - DNA, 40 bp, synthetic construct
  misc_feature 1..40: note = Artificial aptamer TMJ-7
  source 1..40: mol_type = other DNA
gacgacggggttcctccgcggtggggcttgataagtcgtc

SEQ ID NO: 5 - DNA, 42 bp, synthetic construct
  misc_feature 1..42: note = Artificial aptamer TMJ-5
  source 1..42: mol_type = other DNA
gacgacgggatcctccgcggtggggtaacttgatacgtcgtc

SEQ ID NO: 6 - DNA, 42 bp, synthetic construct
  misc_feature 1..42: note = Artificial aptamer TMJ-8
  source 1..42: mol_type = other DNA
gacgacgggatcctccgcggtggggtaacttggtacgtcgtc

SEQ ID NO: 7 - DNA, 41 bp, synthetic construct
  misc_feature 1..41: note = Artificial aptamer TMJ-10
  source 1..41: mol_type = other DNA
gacgacgtcggtcgctccgcgctggggttttagtcgtcgtc

SEQ ID NO: 8 - DNA, 42 bp, synthetic construct
  misc_feature 1..42: note = Artificial aptamer TMJ-11
  source 1..42: mol_type = other DNA
gacgacggtcgctccgcgctggggtgaaattattcagtcgtc

SEQ ID NO: 9 - DNA, 43 bp, synthetic construct
  misc_feature 1..43: note = Artificial aptamer TMJ-4
  source 1..43: mol_type = other DNA
gacgactggggttcctacgcggtgggtctttagctggtcgtcc

SEQ ID NO: 10 - DNA, 44 bp, synthetic construct
  misc_feature 1..44: note = Artificial aptamer TMJ-6
  source 1..44: mol_type = other DNA
gacgacgggtttgttatgagacattgcctaaaggtcgaagctgt

SEQ ID NO: 11 - DNA, 44 bp, synthetic construct
  misc_feature 1..44: note = Artificial aptamer TMJ-9
  source 1..44: mol_type = other DNA
gacgacggatgaagggggattttcgccgtttgctatgaaggagt

SEQ ID NO: 12 - DNA, 42 bp, synthetic construct
  misc_feature 1..42: note = Artificial aptamer AT5-1
  source 1..42: mol_type = other DNA
gacgacgcccgttccaaacttgtcgtgctcttttgtgtcgtc

SEQ ID NO: 13 - DNA, 42 bp, synthetic construct
  misc_feature 1..42: note = Artificial aptamer AT5-2
  source 1..42: mol_type = other DNA
gacgacggagttgcaaacgtcgcggtaggttccccagtcgtc

SEQ ID NO: 14 - DNA, 42 bp, synthetic construct
  misc_feature 1..42: note = Artificial aptamer AT5-3
  source 1..42: mol_type = other DNA
gacgacgcaacagtgcaatacaatggaacgcgacctgtcgtc

SEQ ID NO: 15 - DNA, 42 bp, synthetic construct
  misc_feature 1..42: note = Artificial aptamer AT5-4
  source 1..42: mol_type = other DNA
gacgaccatgagtcccctaagttattaagggtgttcgtcgtc

SEQ ID NO: 16 - DNA, 42 bp, synthetic construct
  misc_feature 1..42: note = Artificial aptamer AT5-5
  source 1..42: mol_type = other DNA
gacgacgtggagcgcatgtgttgctcatctccgatagtcgtc

SEQ ID NO: 17 - DNA, 42 bp, synthetic construct
  misc_feature 1..42: note = Artificial aptamer AT5-6
  source 1..42: mol_type = other DNA
gacgacgcaacaggtaacaactagataccgcgacctgtcgtc

SEQ ID NO: 18 - DNA, 42 bp, synthetic construct
  misc_feature 1..42: note = Artificial aptamer AT5-7
  source 1..42: mol_type = other DNA
gacgacggcatggcgcttgtattgctcaaccttgacgtcgtc

SEQ ID NO: 19 - DNA, 42 bp, synthetic construct
  misc_feature 1..42: note = Artificial aptamer AT5-8
  source 1..42: mol_type = other DNA
gacgacgatcaggagggttccggtggaggtagctaagtcgtc

SEQ ID NO: 20 - DNA, 42 bp, synthetic construct
  misc_feature 1..42: note = Artificial aptamer AT5-9
  source 1..42: mol_type = other DNA
gacgacgatcaggatgttgttataaccggtcgctaagtcgtc

SEQ ID NO: 21 - DNA, 42 bp, synthetic construct
  misc_feature 1..42: note = Artificial aptamer AT5-10
  source 1..42: mol_type = other DNA
gacgacgaccagaaggaatccaagggaggtggctaagtcgtc